(19) United States
(12) Patent Application Publication
(10) Pub. No.: US 2025/0235637 A1
(43) Pub. Date: Jul. 24, 2025

(54) SYSTEMS AND METHODS FOR WIRELESS HUMIDIFIER HEATING FOR INSUFFLATION

(71) Applicant: Intuitive Surgical Operations, Inc., Sunnyvale, CA (US)

(72) Inventors: Noe QUINTERO, Fremont, CA (US); John F. GOODMAN, Ann Arbor, MI (US)

(73) Assignee: Intuitive Surgical Operations, Inc., Sunnyvale, CA (US)

(21) Appl. No.: 19/025,237

(22) Filed: Jan. 16, 2025

Related U.S. Application Data
(60) Provisional application No. 63/622,890, filed on Jan. 19, 2024.

(51) Int. Cl. A61M 13/00

(52) U.S. Cl. CPC A61M 13/003; A61M 2205/368

(57) ABSTRACT

A medical device includes a tube set for use in an insufflation device and that includes a housing defining a reservoir, an inlet port, and an outlet port. The inlet port and the outlet port each are in fluid communication with the reservoir. The inlet port is configured to be coupled to a source of insufflation gas to produce a flow of insufflation gas through the reservoir, and the reservoir is configured to hold a volume of liquid. A conductive member is coupled to the housing and configured to be heated by energy transferred from an inductive coil placed proximate to the housing to heat the volume of liquid to increase a humidity of the insufflation gas flowing through the reservoir. The housing of the tube set is configured to be removably coupled to an insufflation device.

### CROSS-REFERENCE TO RELATED APPLICATIONS

This patent application claims priority to and the filing date benefit of U.S. Provisional Patent Application No. 63/622,890, filed Jan. 19, 2024, entitled “SYSTEMS AND METHODS FOR WIRELESS HUMIDIFIER HEATING FOR INSUFFLATION,” which is incorporated by reference herein in its entirety.

### BACKGROUND

The embodiments described herein relate to medical instruments, and more specifically to medical instruments and methods related to producing heat for a humidifier of an insufflation device, which can be used in minimally-invasive surgical procedures.

A surgical procedure, such as a minimally-invasive surgical procedure, may involve insufflation of a portion of the body with a gas. For example, in a laparoscopic procedure, an insufflation gas may be delivered to the peritoneal cavity of a patient to distend the abdomen, which may improve visual and physical access to internal organs in the abdomen. For example, distension of the patient's abdomen may provide sufficient operating space to enable adequate visualization of the structures and manipulation of instruments inside a patient.

Minimally-invasive laparoscopic surgical procedures may employ surgical systems that operate at least in part with computer-assisted control (“telesurgical systems” or “teleoperated surgical systems”). Such systems are sometimes referred to as robotic surgical systems or surgical robots. The da Vinci® Surgical Systems commercialized by Intuitive Surgical, Inc. are examples of such telesurgical systems.

Various telesurgical system architectures exist, including architectures that enable multiple surgical instruments to enter the body through a single body-opening, sometimes referred to as “single-port” systems (e.g., the da Vinci SP® Surgical System), and architectures that enable multiple surgical instruments to enter the body individually at corresponding multiple locations, sometimes referred to as “multi-port” systems (e.g., the da Vinci Xi® Surgical System). In both the “single-port” and “multi-port” systems, surgical instruments commonly access a body cavity of a patient via one or more cannulas. Single-port systems typically include a cannula mounted to a wound retractor disposed in an incision in the body, while multi-port systems usually have cannulas directly inserted through the body wall/incision. The cannula may also receive or be coupled to a surgical instrument access/seal device, which is configured to seal the opening in the body of the patient and to allow sealed entry/access to the body cavity by surgical instruments. Such instrument access/seal devices may include insufflation fittings for coupling the instrument access/seal device to a source of insufflation gas, which is employed to keep the cavity inflated throughout the surgical procedure.

Prior to employing such teleoperated surgical instruments in a procedure initiated via the abdomen or other body cavity, the surgeon or other clinician needs to establish pneumoperitoneum in the body cavity of the patient. Establishing pneumoperitoneum may involve a number of different approaches to laparoscopic access, including the Veress needle technique (also referred to as “Closed Entry”). The Veress needle technique generally includes inserting a Veress needle (sometimes referred to as Veres needle) into the peritoneal cavity and coupling the proximal (outside the body) end of the needle to an insufflation gas line, which supplies insufflation gas to inflate the body cavity.

Because introduction of relatively dry (or cold) insufflation gas can cause complications, the use of humidified, warm gas for insufflation is desirable. Accordingly, some known insufflation devices use disposable humidifying tube sets to heat and deliver the humidified insufflation gas to a body cavity. The liquid used in the humidifying tube set (e.g., saline or distilled water) is heated to achieve relative humidity that is greater than 95%. Adding heaters and sensors to the tube set, however, greatly increases the cost and complexity to the disposable tube set. In some devices, a heating element is included in the chamber of the tube set and is used to heat the chamber and liquid therein (such as saline or distilled water), but such configurations add complexity and cost to the tube set. For example, in some devices, a resistive heater is used that requires a physical electrical contact between the heater and the power source or controls and/or with the liquid in the chamber. Such designs can be complex and need to be hermetic. In another example, conductive heating can be used, but this requires firm contact against a hot surface, which can be difficult to maintain during use (e.g., due to normal manufacturing variability between parts, differences in how operators use the device, etc.). It is desirable to simplify and reduce the cost of such tube sets to reduce the overall cost of the surgery.

Thus, a need exists to reduce the cost of disposable tube sets while at the same time provide sufficient heating and humidifying capabilities.

### SUMMARY

This summary introduces certain aspects of the embodiments described herein to provide a basic understanding. This summary is not an extensive overview of the inventive subject matter, and it is not intended to identify key or critical elements or to delineate the scope of the inventive subject matter.

In some embodiments, a tube set is described for use in an insufflation device and includes a housing defining a reservoir, an inlet port, and an outlet port. The inlet port and the outlet port each are in fluid communication with the reservoir. The inlet port is configured to be coupled to a source of insufflation gas to produce a flow of insufflation gas through the reservoir, and the reservoir is configured to hold a volume of liquid. A conductive member is coupled to the housing and is configured to be heated by energy transferred from an inductive coil placed proximate to the housing to heat the volume of liquid to increase a humidity of the insufflation gas flowing through the reservoir.

In some embodiments, the housing of the tube set is configured to be removably coupled to an insufflation device. In some embodiments, the conductive member is positioned to be aligned with an inductive coil of the insufflation device. In some embodiments, the conductive member is a plate having a planar configuration. In some embodiments, the conductive member is physically spaced apart from the inductive coil of the insufflation device.

In some embodiments, the tube set includes a tube coupled to the outlet and configured to convey humidified insufflation gas from within the reservoir to a location within a patient's body. In some embodiments, the tube is devoid of a heating element. In some embodiments, the tube set includes a liquid port configured to be coupled to a source of liquid to be introduced into the reservoir. In some embodiments, a filter is disposed within the reservoir and configured to transfer a portion of the volume of liquid disposed within the housing to a different location within the housing to provide humified fluid within the reservoir. In some embodiments, the conductive member is disposed within the reservoir and positioned adjacent to a wall of the housing.

In some embodiments, the tube set includes a diffuser disposed within the reservoir that is configured to allow insufflation gas to flow through the diffuser and into the liquid within the reservoir. In some embodiments, the diffuser includes holes or perforations to allow insufflation gas to flow through the diffuser. In some embodiments, the diffuser is a porous structure configured to allow insufflation gas to flow through the diffuser.

In some embodiments an insufflation system includes an insufflator housing configured to removably receive a humidifying tube set couplable within an interior of the insufflator housing. The humidifying tube set includes a tube set housing defining a reservoir and a conductive member coupled to the tube set housing. An inductive coil heater is positioned to heat the conductive member of the humidifying tube set when the humidifying tube set is coupled within the insufflator housing. A controller is coupled to the inductive coil heater.

In some embodiments, a temperature sensor is positioned within the insufflator housing to measure a temperature of the humidifying tube set when the humidifying tube set is coupled within the insufflator housing. In some embodiments, the temperature sensor is disposed spaced apart from the humidifying tube set when the humidifying tube set is coupled within the housing. In some embodiments, the temperature sensor includes an infrared sensor.

In some embodiments, the conductive member is disposed within the reservoir and positioned adjacent to a wall of the tube set housing. In some embodiments, the inductive coil heater is configured to heat the conductive member of the humidifying tube set to heat a liquid within the reservoir of the humidifying tube set and create a relative humidity within the humidifying tube set of greater than 95%.

In some embodiments, the insufflation system includes a source of insufflation gas couplable to an inlet of the humidifying tube set. In some embodiments, a volume of the insufflation gas can flow through the inlet of the humidifying tube set, be heated within the reservoir of the humidifying tube set by the inductive coil heater and exit an outlet of the humidifying tube set at a relative humidity of greater than 95%. In some embodiments, the controller is configured to determine a relative humidity of an insufflation gas within the reservoir based at least in part on a temperature measurement taken with the temperature sensor.

### BRIEF DESCRIPTION OF THE DRAWINGS

FIG. 1A depicts a surgeon accessing the abdominal cavity of a patient via a Veress needle technique.

FIG. 1B illustrates an example insufflation device and tube set, according to an embodiment.

FIG. 1C illustrates the insufflation device of FIG. 1B with a portion of the tube set prior to being inserted into an interior of the insufflation device.

FIG. 2 is a schematic illustration of an insufflation system according to an embodiment.

FIG. 3 is a top view illustration of a portion of a controller and an inductive coil heater of an insufflation system, according to an embodiment.

FIG. 4 is a schematic illustration of a humifying tube set for use in an insufflation device, according to an embodiment.

FIG. 5 is a schematic illustration of a portion of an insufflation device, according to an embodiment.

FIG. 6 is a perspective view of a portion of a humidifying tube set according to an embodiment, shown with the tube set housing transparent for illustration purposes.

FIG. 7 is an exploded view of the humidifying tube set of FIG. 6.

FIG. 8 is a graph illustrating power output versus distance between an inductive coil heater of an insufflation device and a conductive member of a humidifying tube set as described herein.

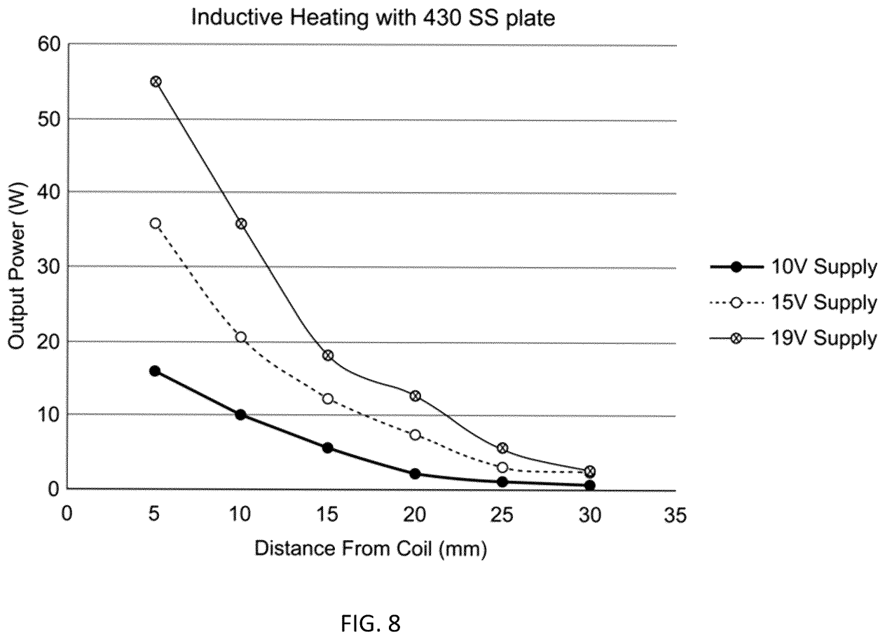

FIG. 9 is a schematic illustration of a portion of a humidifying tube set, according to another embodiment.

FIG. 10 is a perspective view of a humidifying tube set, according to another embodiment, shown with the tube set housing transparent for illustration purposes.

FIG. 11 is a side view of the humidifying tube set of FIG. 10.

FIG. 12 is an exploded view of the humidifying tube set of FIG. 10.

FIG. 13 is a front perspective view of a humidifying tube set, according to another embodiment, shown with the tube set housing transparent for illustration purposes.

FIG. 14 is a rear perspective view of the humidifying tube set of FIG. 13.

FIG. 15 is an exploded view of the humidifying tube set of FIG. 13.

FIG. 16 is a top perspective view of the bottom housing portion of the humidifying tube set of FIG. 13.

### DETAILED DESCRIPTION

As described above, some known insufflation devices use disposable humidifying tube sets to heat and deliver an insufflation gas (e.g., CO₂) to a body cavity. The liquid within the humidifying tube set (e.g., saline or distilled water) is heated to achieve a desired relative humidity (e.g., greater than 95%). Adding heaters and sensors to the tube set greatly increases the cost and complexity to the disposable tube set. Thus, devices and methods described herein provide a simplified and reduced cost humidifying tube set and insufflation device to reduce the overall cost of an insufflation system and the corresponding surgery in which it is used. For example, the devices and methods described herein reduce (or eliminate) the physical connections between the components of the insufflation system (e.g., between the heaters with direct exposure to the liquid and the electronic controls or power supply), which can reduce the likelihood of leaks (between interfacing components). The devices and methods described herein are also easy to use, having minimal steps for alignment, insertion and removal of the disposable components (e.g., the tube set). The devices and methods described herein also accommodate variation in parts (e.g., due to normal manufacturing tolerances) while still providing efficient heating of the liquid to provide the desired humidity.

As described herein, in some embodiments, devices and methods for an insufflation system include an inductive coil heater to heat the liquid within a reservoir of a humidifying tube set. The inductive coil heater produces a strong magnetic field in the humidification reservoir within a housing of the humidifying tube set. The overall humidifying tube set is referred to herein as a tube set and includes components such as one or more tubes and a housing defining a reservoir. A conductive member such as a metal plate is positioned inside the reservoir near the coil and the liquid disposed within the reservoir is heated by the inductive heating of the conductive member. This greatly simplifies the tube set assembly and allows for wireless heating of the liquid within the reservoir of the tube set. In some embodiments, a non-contact infrared temperature sensor can be provided to monitor the temperature in the humidification chamber. For example, a temperature reading of a surface of the housing can be measured that can be used to determine the temperature inside the reservoir of the tube set.

As described herein, using inductive heating provided by the insufflation device and a non-contact temperature sensor included in the insufflation device reduces the cost of the disposable tube set by including the heater and sensor in the reusable insufflation system. This allows the heater and sensor to be used more than once and provides for a lower cost disposable tube set.

In some embodiments, rather than a heat plate within the humidifying cartridge, the housing of the humidifying cartridge can be formed with a metallic material that can be heated by the inductive coil. In an alternative method for providing a heater to heat the liquid within the humidifying cartridge, radio frequency (RF) waves and microwaves can be used to wirelessly heat the water directly without a plate. In such an embodiment, a faraday cage can be used to surround the cartridge to protect against escaping RF energy.

As used herein, the term “about” when used in connection with a referenced numeric indication means the referenced numeric indication plus or minus up to 10 percent of that referenced numeric indication. For example, the language “about 50” covers the range of 45 to 55. Similarly, the language “about 5” covers the range of 4.5 to 5.5.

As used in this specification and the appended claims, the word “distal” refers to direction towards a work site, and the word “proximal” refers to a direction away from the work site. Thus, for example, the end of a tool that is closest to the target tissue would be the distal end of the tool, and the end opposite the distal end (i.e., the end manipulated by the user or coupled to the actuation shaft) would be the proximal end of the tool.

Further, specific words chosen to describe one or more embodiments and optional elements or features are not intended to limit the invention. For example, spatially relative terms—such as “beneath”, “below”, “lower”, “above”, “upper”, “proximal”, “distal”, and the like—may be used to describe the relationship of one element or feature to another element or feature as illustrated in the figures. These spatially relative terms are intended to encompass different positions (i.e., translational placements) and orientations (i.e., rotational placements) of a device in use or operation in addition to the position and orientation shown in the figures. For example, if a device in the figures is turned over, elements described as “below” or “beneath” other elements or features would then be “above” or “over” the other elements or features. Thus, the term “below” can encompass both positions and orientations of above and below. A device may be otherwise oriented (e.g., rotated 90 degrees or at other orientations) and the spatially relative descriptors used herein interpreted accordingly. Likewise, descriptions of movement along (translation) and around (rotation) various axes include various spatial device positions and orientations. The combination of a body's position and orientation defines the body's pose.

Similarly, geometric terms, such as “parallel”, “perpendicular”, “round”, or “square”, are not intended to require absolute mathematical precision, unless the context indicates otherwise. Instead, such geometric terms allow for variations due to manufacturing or equivalent functions. For example, if an element is described as “round” or “generally round,” a component that is not precisely circular (e.g., one that is slightly oblong or is a many-sided polygon) is still encompassed by this description.

In addition, the singular forms “a”, “an”, and “the” are intended to include the plural forms as well, unless the context indicates otherwise. The terms “comprises”, “includes”, “has”, and the like specify the presence of stated features, steps, operations, elements, components, etc. but do not preclude the presence or addition of one or more other features, steps, operations, elements, components, or groups.

Unless indicated otherwise, the terms apparatus, medical device, instrument, and variants thereof, can be interchangeably used.

Inventive aspects are described with reference to a teleoperated surgical system. An example architecture of such a teleoperated surgical system is the da Vinci® surgical system commercialized by Intuitive Surgical, Inc., Sunnyvale, California. Knowledgeable persons will understand, however, that inventive aspects disclosed herein may be embodied and implemented in various ways, including computer-assisted, non-computer-assisted, and hybrid combinations of manual and computer-assisted embodiments and implementations. Implementations are merely presented as examples, and they are not to be considered as limiting the scope of the inventive aspects disclosed herein. As applicable, inventive aspects may be embodied and implemented in both relatively smaller, hand-held, hand-operated devices and relatively larger systems that have additional mechanical support.

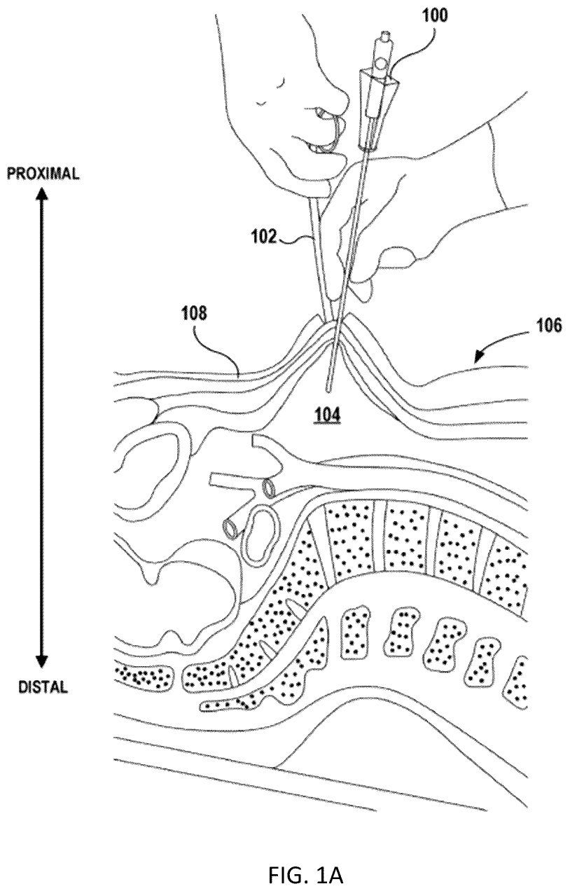

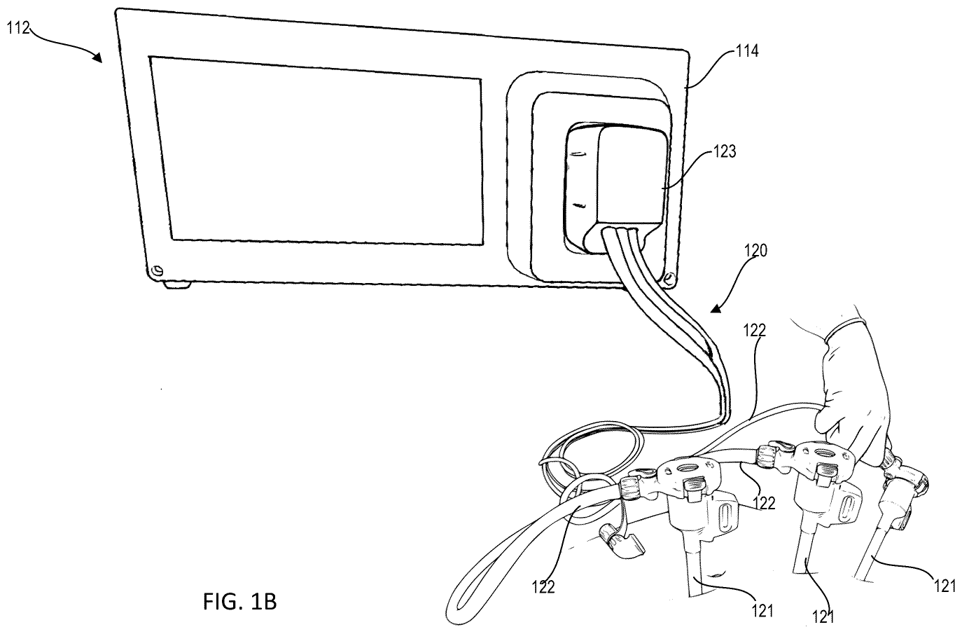

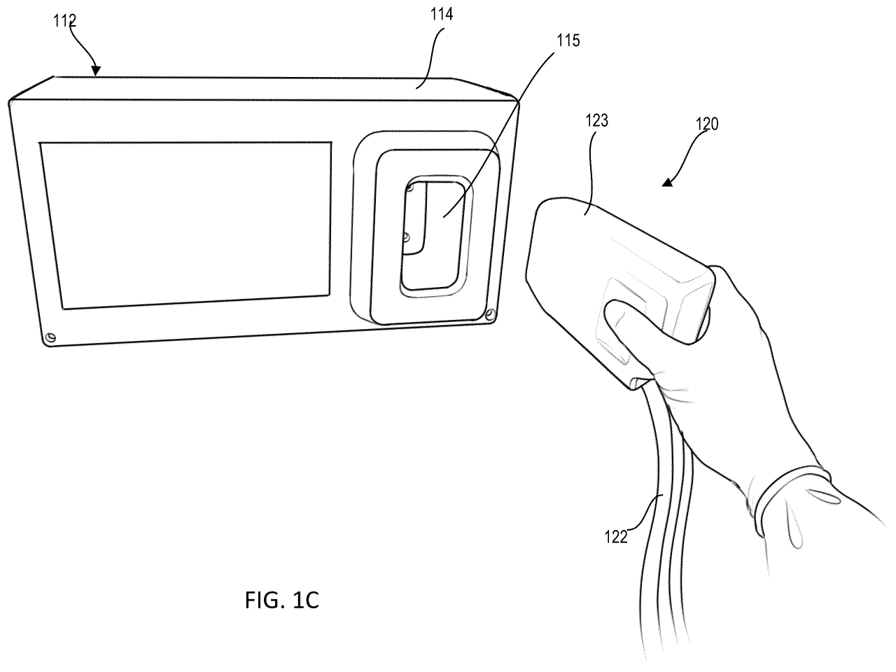

FIG. 1A depicts an example procedure of accessing a body cavity of a patient and providing an insufflation gas to the cavity. FIG. 1A, illustrates a surgeon accessing an abdominal cavity of a patient via an example of the Veress needle technique and the process of providing an insufflation gas to the cavity. In FIG. 1A, Veress needle 100 and forceps 102 are employed to access the abdominal (peritoneal) cavity 104 of patient 106 through body wall 108. Forceps 102 may be employed to grasp and fix the skin of body wall 108 advantageously for a superficial incision and insertion of Veress needle 100.

An insufflation line (not shown) can be connected to the proximal end of Veress needle 100 and an insufflation gas (e.g., CO₂) can be pumped through the insufflation line and Veress needle 100 into peritoneal cavity 104 at a relatively low initial pressure (e.g., 10 millimeters of mercury-mmHg), after which the insufflation pressure can be raised (e.g., to 15 mmHg) to inflate the cavity.

Veress needle 100 is an example of a “pneumocavity” needle. As described herein, a “pneumocavity” needle is a needle used to introduce pneumotosis into a body cavity to create space. Although examples described in this disclosure reference the Veress Needle Technique and use of a Veress needle, examples in accordance with this disclosure can also be employed with other “pneumocavity” needles and employed in other types of procedures.

As described herein, in some cases, an insufflation system can be used to provide an insufflation gas (e.g., carbon dioxide (CO₂) to a body cavity. The insufflation system can include an insufflation device that can removably receive a disposable tube set to be used to deliver an insufflation gas to the body cavity. FIGS. 1B and 1C illustrate an example insufflation device 112 and a disposable tube set 120. The insufflation device 112 includes an insufflator housing 114 defining an interior region 115 (shown in FIG. 1C) that can receive the disposable tube set cartridge 120. The insufflation device 112 can also include any suitable components to perform the functions described here, such as, for example, valves, gas lines, fluidic couplings, sensors, and any electronic components or circuits for controlling the delivery of insufflation gas. The insufflation device 112 and tube set cartridge 120 can be coupled to a source of insufflation gas (not shown in FIGS. 1B and 1C), such as a source of CO₂. The tube set cartridge 120 can include one or more delivery tubes 122 that can be coupled to a medical device 121 to deliver the insufflation gas to a cavity within the patient's body. For example, the medical device 121, can be a cannula, a Veress needle described for FIG. 1A or other suitable device to deliver the insufflation gas to the body cavity. The tube set 120 can include a tube set (or cartridge) housing 123 that defines a reservoir (not shown in FIGS. 1B and 1C) and can be removably received within the interior region 115 of the housing 114 of the insufflation device 112. The reservoir of the tube set housing 123 can be coupled to the source of insufflation gas such that insufflation gas can pass through the reservoir of the tube set housing 120, through one or more delivery tubes 122, through one or more of the medical devices 121 and into the body cavity of the patient. The reservoir of the tube set 120 can also hold a volume of liquid, such as saline or distilled water, which can be heated within the reservoir of the tube set housing 123. This in turn heats and humidifies the insufflation gas passing through the reservoir and provides humidified insufflation gas to the body cavity. For example, in some cases, it may be desirable to provide insufflation gas at greater than 95% relative humidity to the body cavity. The liquid can be provided via a source of liquid (not shown in FIG. 1B) that can be coupled to the reservoir of the tube set housing 123 when the tube set 120 is coupled to the insufflation device 112.

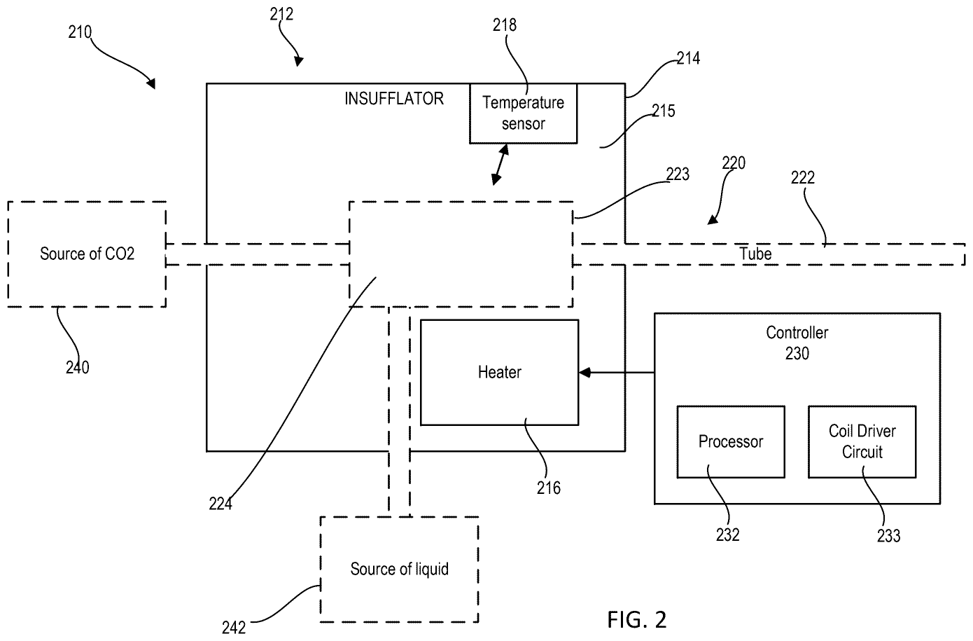

FIG. 2 is a schematic illustration of an insufflation system, according to an embodiment. An insufflation system 210 includes an insufflation device 212 and a controller 230. As described herein, in some cases, an insufflation system can be used to provide an insufflation gas (e.g., CO₂) to a body cavity. The insufflation device 212 includes an insufflator housing 214, a heater 216 and a temperature sensor 218. The controller 230 includes a processor 232 and a coil driver circuit 233 used to control the heater 216. Although shown schematically as being outside of the insufflator housing 214, in some embodiments, the controller 230 can be within the insufflator housing 214. In other embodiments, the controller 230 can be outside of the insufflator housing 214 and can be operatively coupled to the heater 216, the temperature sensor 218 or any other electronic components of the insufflation system 210 by any suitable mechanism (e.g., wired or wireless).

The insufflator housing 214 defines an interior region 215 that can removably receive a humidifying tube set 220 to deliver an insufflation gas to a body cavity as described above with respect to FIG. 1A-1C. The tube set 220 can be similar to (or include the same features as) any of the tube sets shown and describe herein. The tube set 220 can include a tube set housing 223 that defines a reservoir 224 that can contain a liquid and that receives a flow of insufflation gas such that the liquid can be heated to humidify the insufflation gas. The tube set 220 can include one or more delivery tubes 222 that can be coupled to a delivery instrument (not shown) used to deliver insufflation gas to a body cavity as described herein. In some embodiments, the tube set 220 (and any of the tube sets described herein) can be configured as a disposable, single-use component of the system. Accordingly, in some embodiments, the tube set 220 is configured to be discarded after use via standard, non-regulated waste procedures. In other embodiments, the tube set 220 can be discarded via standard medical waste procedures.

The temperature sensor 218 can be positioned within the interior region 215 of the insufflator housing 214 and can be used to measure a temperature of the humidifying tube set 220 (including the water therein, the temperature of the gas flowing therethrough, or any other aspects of the tube set) when the humidifying tube set 220 is coupled within the insufflator housing 214. In some embodiments, the temperature sensor 218 is disposed at a spaced distance from the humidifying cartridge 220 when the humidifying tube set 220 is coupled within the insufflator housing 214. Thus, the temperature sensor 218 does not have to contact the humidifying cartridge 220. In some embodiments, the temperature sensor 218 includes an infrared sensor.

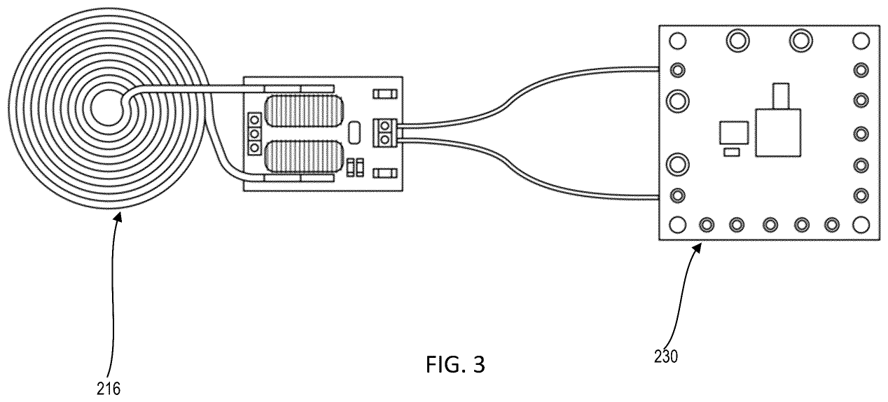

The heater 216 can be an inductive coil heater, as shown in FIG. 3, which illustrates the heater 216 operably coupled to the controller 230. The heater 216 can be positioned near a conductive member (not shown in FIG. 3) of the humidifying tube set 220. For example, the conductive member can be coupled to a housing (not shown in FIG. 3) of the humidifying tube set 220, as described in more detail herein. In some embodiments, the conductive member is coupled to a wall of the tube set housing. In some embodiments, the conductive member is positioned to be aligned with the inductive coil of the heater 216 of the insufflation device 212. In some embodiments, the conductive member is a plate having a planar configuration. In some embodiments, the conductive member is physically spaced apart from the inductive coil of the heater 216 of the insufflation device.

The heater 216 is configured to heat the conductive member of the humidifying cartridge 220 by electromagnetic induction currents that are generated by the induction coils of the heater 216. The currents induced in the conductive member cause the conductive member to heat, thereby heating a liquid (e.g., saline or distilled water) within the reservoir (not shown in FIG. 2) of the humidifying tube set 220. The heater 216 can heat the liquid, which facilitates creating a desired relative humidity of the insufflation gas within (or passing through) the reservoir of the humidifying cartridge. For example, in some embodiments, the heater 216 can create a relative humidity of greater than 95%. In some embodiments, a diffuser can be used to increase the contact time of the gas to the liquid, and increase the efficiency of the humidification process.

A source of insufflation gas 240, such as a source of CO₂, can be coupled to the cartridge 220 when the cartridge 220 is coupled to the insufflator housing 214 of the insufflation device 212. As discussed above, the cartridge 220 can include one or more delivery tubes 222 that can be coupled to a delivery instrument (not shown), used to deliver the insufflation gas to a body cavity. A source of liquid 242 can also be coupled to (or included within) the cartridge 220 when the cartridge 220 is coupled to the insufflator housing 214 of the insufflation device 212. The source of liquid can provide a volume of liquid, such as saline or distilled water, to the interior reservoir of the cartridge housing. As described above, the heater 216 is used to heat the volume of liquid within the cartridge 220 to heat and humidify the insufflation gas to be delivered to the body cavity.

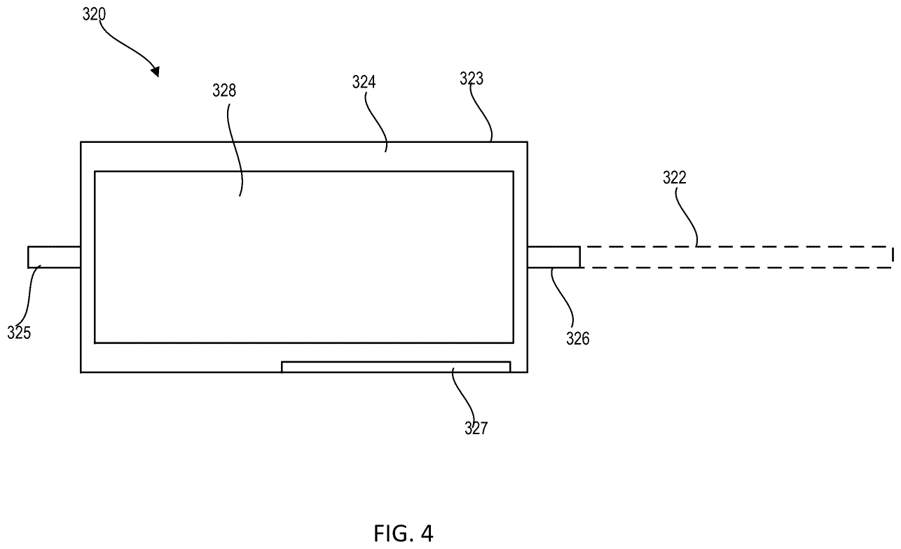

FIG. 4 is a schematic illustration of a humidifying tube set 320 according to an embodiment. The humidifying tube set 320 can be coupled to an insufflation device as described herein, such as, for example, insufflation device 212. The tube set 320 can include a tube set housing 323 that defines a reservoir 324 and can be removably received within an interior region (e.g., 215) of the housing (e.g., 214) of an insufflation device (e.g., 212). In some embodiments the housing 323 can include multiple components that when coupled together collectively define the reservoir 324. The tube set 320 can include one or more delivery tubes 322 that can be coupled to a delivery instrument (not shown) used to deliver insufflation gas to a body cavity as described herein. The delivery tube(s) 322 can be coupled to an outlet 326 of the tube set housing 323 which is in fluid communication with the reservoir 324, as shown in FIG. 4. As described above for previous embodiments, the reservoir 324 of the cartridge housing 323 can be coupled to a source of insufflation gas (not shown in FIG. 4) such that insufflation gas can pass through the reservoir 324 of the tube set housing 323, through one or more delivery tubes 322 and into the body cavity of the patient. The source of insufflation gas can be coupled to, for example, an inlet 325 of the tube set housing 323.

The reservoir 324 can also hold a volume of liquid, such as saline or distilled water, which can be heated within the reservoir 324 to heat and humidify the insufflation gas passing through the reservoir 324 and provide humidified insufflation gas to the body cavity. For example, in some cases, it may be desirable to provide insufflation gas having greater than 95% relative humidity to the body cavity. The liquid can be provided via a source of liquid (not shown in FIG. 4) that can be coupled to the reservoir of the tube set housing 323 when the cartridge 320 is coupled to the insufflation device.

The tube set 320 also includes a conductive member 327 coupled to the housing 323 and configured to be heated by energy transferred from an inductive coil heater positioned proximate to the housing 323 to heat the volume of liquid and increase a humidity of the insufflation gas flowing through the reservoir 324. The conductive member 327 can be, for example, a metal plate. In some embodiments, the conductive member 327 is coupled to a wall of the cartridge housing 323. In some embodiments, the conductive member 327 is positioned to be aligned with the inductive coil of the heater of the insufflation device. In some embodiments, the conductive member 327 is a plate having a planar configuration. In some embodiments, the conductive member 327 is physically spaced apart from the inductive coil of the insufflation device 212.

The inductive coil heater (e.g., 216 described above) is configured to heat the conductive member 327 to heat the volume of liquid (e.g., saline or distilled water) within the reservoir 324 of the cartridge 320. The cartridge 320 also includes one or more filters 328 within the reservoir 324. The filters 328 can be a single construction of multiple filters, a single filter, or multiple separate filters. In some embodiments, the filter 328 is a corrugated paper construction. In some embodiments, the filter 328 can be a baffle construction. The filter 328 can be constructed with, for example, a nylon, a sponge material, metal mesh, steel wool, or other porous coalescing media that is biocompatible and does not dissolve when exposed to liquid. The filter 328 is configured to transfer a portion of the volume of liquid disposed within the tube set housing 323 to a location within the housing 323 to provide humified fluid within the reservoir 324.

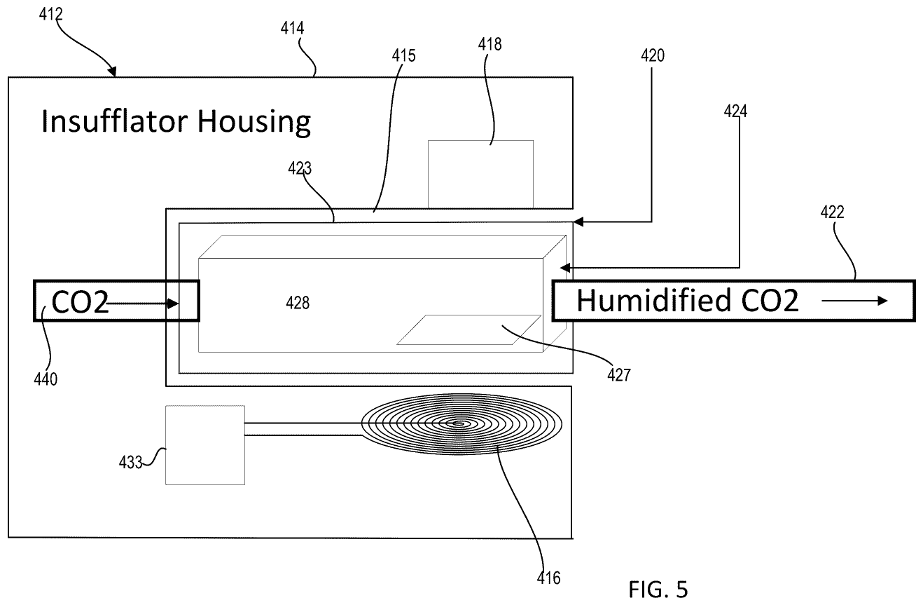

FIG. 5 is a schematic illustration of an insufflation device and humidifying tube set according to another embodiment. An insufflation device 412 can be part of an insufflation system such as insufflation system 210, and used to provide an insufflation gas (e.g., CO₂) to a body cavity. The insufflation device 412 includes an insufflator housing 414 that defines an interior region 424, a heater 416 and a temperature sensor 418. The insufflation device 412 can be communicatively coupled to a controller (e.g., 230) that includes a processor (e.g., 232) and a coil driver circuit 433 used to control the heater 418.

The interior region 415 of the insufflator housing 423 can removably receive a humidifying tube set 420 to deliver an insufflation gas to a body cavity as described herein. The temperature sensor 418 is positioned within the interior region 415 of the insufflator housing 414 and can be used to measure a temperature of the humidifying tube set 420 (including the water therein, the temperature of the gas flowing therethrough, or any other aspects of the tube set) when the humidifying tube set 420 is coupled within the insufflator housing 414. In some embodiments, the temperature sensor 418 is disposed at a spaced distance from the humidifying tube set 420 when the humidifying tube set 420 is coupled within the insufflator housing 414. Thus, the temperature sensor 418 does not have to contact the humidifying tube set 420. In some embodiments, the temperature sensor 418 includes an infrared sensor as shown in FIG. 5.

The heater 416 can be an inductive coil heater, as shown in FIG. 5, which is positioned near a conductive member 427 of the humidifying tube set 420. The heater 416 is operatively coupled to the coil driver circuit 433 of the controller (see also heater 216 and controller 230 in FIG. 3).

The tube set 420 includes a tube set housing 423 that defines a reservoir 424 and can be removably received within the interior region 415 of the housing 414 of the insufflation device 412. The tube set 420 can include one or more delivery tubes 422 (only one tube 422 is shown in FIG. 5) that can be coupled to a delivery instrument (not shown) used to deliver insufflation gas to a body cavity as described herein. The delivery tube(s) 422 can be coupled to an outlet (not shown in FIG. 5) of the tube set housing 423 and in fluid communication with the reservoir 424. As described above for previous embodiments, the reservoir 424 of the tube set housing 423 can be coupled to a source of insufflation gas 440 such that insufflation gas can pass through the reservoir 424 of the tube set housing 423, through one or more delivery tubes 422 and into the body cavity of the patient. The source of insufflation gas can be coupled to, for example, an inlet (not shown in FIG. 5) of the tube set housing 423.

The reservoir 424 can also hold a volume of liquid, such as saline or distilled water, which can be heated within the reservoir 424 to heat and humidify the insufflation gas passing through the reservoir 424 and provide humidified insufflation gas to the body cavity. For example, in some cases, it may be desirable to provide insufflation gas having greater than 95% relative humidity to the body cavity. The liquid can be provided via a source of fluid (not shown in FIG. 5) that can be coupled to the reservoir 424 of the tube set housing 423 when the tube set 420 is coupled to the insufflation device 412.

The tube set 420 also includes the conductive member 427 coupled to the housing 423 and configured to be heated by energy transferred from the inductive coil of the heater 416 positioned proximate to the housing 423 and conductive member 427 to heat the volume of liquid and increase a humidity of the insufflation gas flowing through the reservoir 424. The conductive member 427 can be, for example, a metal plate. In some embodiments, the conductive member 427 is coupled to a wall of the tube set housing 423. In some embodiments, the conductive member 427 is positioned to be aligned with the inductive coil of the heater 416 of the insufflation device 412. In some embodiments, the conductive member 427 is a plate having a planar configuration. In some embodiments, the conductive member 427 is physically spaced apart from the inductive coil of the heater 416 of the insufflation device 412.

The tube set 420 also includes a filter 428 within the reservoir 424. The filter 428 can be a single construction of multiple filters, a single filter, or multiple separate filters. In some embodiments, the filter 428 is a corrugated paper construction. In some embodiments, the filter 428 is a baffle construction. The filter 428 is configured to transfer a portion of the volume of liquid disposed within the tube set housing 423 to a location within the housing 423 to provide humified fluid within the reservoir 424.

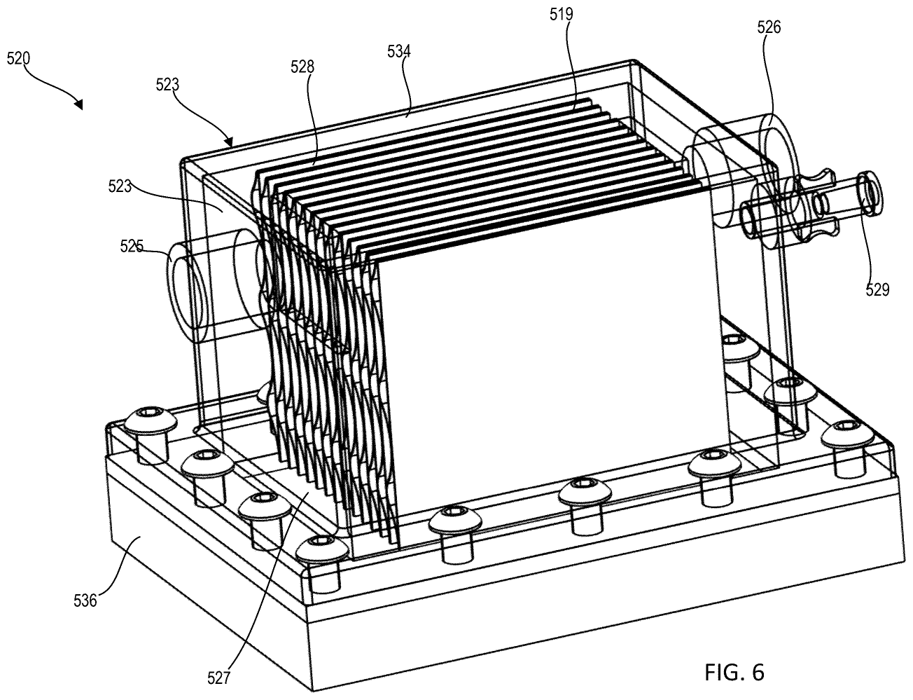

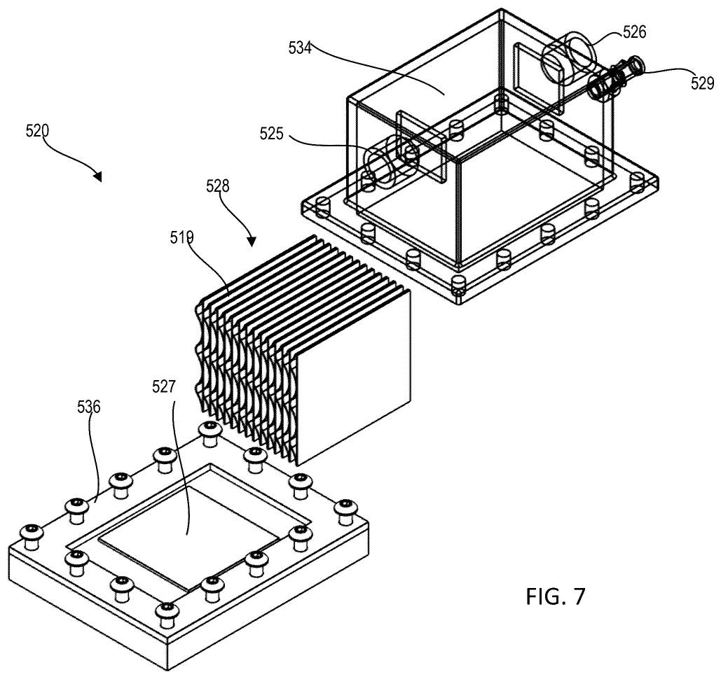

FIGS. 6 and 7 illustrate a humidifying tube set 520 according to another embodiment. The humidifying tube set 520 can be coupled to an insufflation device as described herein, such as insufflation devices 212 or 412. The tube set 520 can include a tube set housing 523 that defines a reservoir 524 and can be removably received within an interior region (e.g., 215) of the housing (e.g., 214) of an insufflation device (e.g., 212). The housing 524 of the tube set 520 includes a top housing portion 534 and a bottom housing portion 536 that collectively define the reservoir 524. The tube set 520 can include one or more delivery tubes (not shown in FIGS. 6 and 7) that can be coupled to a delivery instrument (not shown) used to deliver insufflation gas to a body cavity as described herein. The delivery tube(s) can be coupled to an outlet 526 of the tube set housing 523 and be in fluid communication with the reservoir 524. As described above for previous embodiments, the reservoir 524 of the tube set housing 523 can be coupled to a source of insufflation gas (not shown in FIGS. 6 and 7) such that insufflation gas can pass through the reservoir 524 of the tube set housing 523, through one or more delivery tubes and into the body cavity of the patient. The source of insufflation gas can be coupled to, for example, an inlet 525 of the tube set housing 523.

The reservoir 524 can also hold a volume of liquid, such as saline or distilled water, which can be heated within the reservoir 524 to heat and humidify the insufflation gas passing through the reservoir 524 and provide humidified insufflation gas to the body cavity. For example, in some cases, it may be desirable to provide insufflation gas at greater than 95% relative humidity to the body cavity. The liquid can be provided via a source of liquid (not shown in FIG. 5) that can be coupled to a fill port 529 of the tube set housing 523 when the tube set 520 is coupled to an insufflation device.

The tube set 520 also includes a conductive member 527 coupled to the housing 523 within the reservoir 524 and configured to be heated by energy transferred from an inductive coil of a heater positioned proximate to the housing 523 to heat the volume of liquid and increase a humidity of the insufflation gas flowing through the reservoir 524. The conductive member 527 can be, for example, a metal plate. In some embodiments, the conductive member 527 is coupled to a wall of the tube set housing 523. In this embodiment, the conductive member 527 is disposed on a surface of the bottom housing portion 536. In some embodiments, the conductive member 527 is positioned to be aligned with the inductive coil of the heater of the insufflation device. In some embodiments, the conductive member 527 is a plate having a planar configuration. In some embodiments, the conductive member 527 is physically spaced apart from the inductive coil of the heater of the insufflation device. The inductive coil heater (e.g., 216, 416 described above) is configured to heat the conductive member 527 to heat the volume of liquid (e.g., saline or distilled water) within the reservoir 524 of the tube set 520.

The tube set 520 also includes a filter 528 within the reservoir 524. The filter 528 can include one or more components 519. As shown in FIG. 5, in this embodiment the filter 528 includes multiple filter components 519. As described above, in alternative embodiments, a filter 528 can include a single filter component 519. In some embodiments, the filter 528 is a corrugated paper construction. In some embodiments, the filter 528 is a baffle construction. In some embodiments, the filter 528 is configured to transfer a portion of the volume of liquid disposed within the tube set housing 523 to a location within the housing 523 to provide humified fluid within the reservoir 524. For example, as the volume of liquid within the reservoir 524 is heated, the liquid will collect on the filter 528, and as the insufflation gas flows through the reservoir 524, the insufflation gas will be heated and humidified as it passes through the filters 528. In some embodiments, a filter such as filter 528 is not used. In some embodiments, a diffuser (not shown in FIGS. 5 and 6) is provided with or without a filter. The diffuser can cause a bubbling action of the insufflation gas through the liquid within the reservoir, which can increase the contact time of the insufflation gas to the liquid within the reservoir, and increase the efficiency of the humidification process.

FIG. 7 is a graph illustrating power output versus a distance between an inductive coil heater of an insufflation device and a conductive member of a humidifying tube set as described herein. The graph illustrates the power output versus distance for three different supply voltages. As shown in the graph, the position of the inductive coil heater relative to the conductive member impacts the power output to provide inductive heating of the liquid within the tube set. For example, the shorter the distance between the inductive coil and the conductive member, the higher the power output.

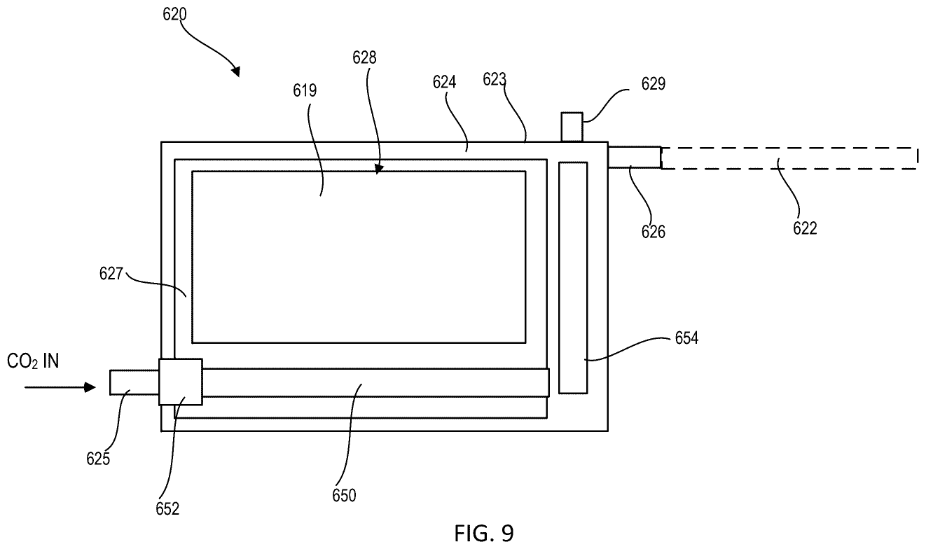

FIG. 9 is a schematic illustration of a humidifying tube set 620 according to an embodiment. The humidifying tube set 620 can be coupled to an insufflation device as described herein, such as, for example, insufflation device 212. The tube set 620 can include a tube set housing 623 that defines a reservoir 624 and can be removably received within an interior region (e.g., 215) of the housing (e.g., 214) of an insufflation device (e.g., 212). In some embodiments the housing 623 can include multiple components that when coupled together collectively define the reservoir 624. The tube set 620 can include one or more delivery tubes 622 that can be coupled to a delivery instrument (not shown) used to deliver insufflation gas to a body cavity as described herein. The delivery tube(s) 622 can be coupled to an outlet 626 of the tube set housing 623 which is in fluid communication with the reservoir 624, as shown in FIG. 9. As described above for previous embodiments, the reservoir 624 of the tube set housing 623 can be coupled to a source of insufflation gas (not shown in FIG. 9) such that insufflation gas can pass through the reservoir 624 of the tube set housing 623, through one or more delivery tubes 622 and into the body cavity of the patient. The source of insufflation gas can be coupled to, for example, an inlet port 625 of the tube set housing 623.

The reservoir 624 can also hold a volume of liquid, such as saline or distilled water, which can be heated within the reservoir 624 to heat and humidify the insufflation gas passing through the reservoir 624 and provide humidified insufflation gas to the body cavity. For example, in some cases, it may be desirable to provide insufflation gas having greater than 95% relative humidity to the body cavity. The liquid can be provided via a source of liquid (not shown in FIG. 9) that can be coupled to the reservoir of the tube set housing 623 when the tube set 620 is coupled to the insufflation device. For example, the housing 623 can include a liquid fill port 629 that can be coupled to a source of liquid such as a saline bag with tubing. In some embodiments, a float valve 654 is coupled near the liquid fill port 629 to regulate the flow of liquid into the reservoir 624.

The tube set 620 also includes a conductive member 627 coupled to the housing 623. The conductive member 627 is configured to be heated by energy transferred from an inductive coil heater positioned proximate to the housing 623 to heat the volume of liquid and increase a humidity of the insufflation gas flowing through the reservoir 624. The conductive member 627 can be, for example, a metal plate. In some embodiments, the conductive member 627 is coupled to a wall of the tube set housing 623. In some embodiments, the conductive member 627 is positioned to be aligned with the inductive coil of the heater of the insufflation device. In some embodiments, the conductive member 627 is a plate having a planar configuration. In some embodiments, the conductive member 627 is physically spaced apart from the inductive coil of the insufflation device. As shown in FIG. 9, the conductive member 627 is disposed on a wall of the housing 623.

The tube set 620 can also include one or more one-way valves 652 at the inlet port 625 that can be used to prevent insufflation gas and/or liquid from within the reservoir 624 from flowing back out through the inlet port 625. In some embodiments, the one-way valve can be for example, an umbrella valve.

The inductive coil heater (e.g., 216 described herein) is configured to heat the conductive member 627 to heat the volume of liquid (e.g., saline or distilled water) within the reservoir 624 of the tube set 620. The tube set 620 also includes a filter 628 within the reservoir 624. The filter 628 can be a single construction of multiple filters, a single filter, or multiple separate filters. As described for previous embodiments, the filter 628 can include one or more filter components 619. In some embodiments, the filter 628 is a corrugated paper construction. In some embodiments, the filter 628 can be a baffle construction. The filter 628 can be constructed with, for example, a nylon, a sponge material, metal mesh, steel wool, or other porous coalescing media that is biocompatible and does not dissolve when exposed to liquid. The filter 628 is configured to transfer a portion of the volume of liquid disposed within the tube set housing 623 to a location within the housing 623 to provide humified fluid within the reservoir 624. For example, as the volume of liquid within the reservoir 624 is heated, the liquid will collect on the filter 628, and as the insufflation gas flows through the reservoir 624, the insufflation gas will be heated and humidified as it passes through the filter 628.

The tube set 620 can also include one or more diffuser 650 as shown in FIG. 9. The diffuser 650 allows insufflation gas to flow from the inlet port 625, through the diffuser 650, and into the liquid within the reservoir 624. The diffuser 650 causes a bubbling action of the insufflation gas through the liquid within the reservoir 624, which can increase the contact time of the insufflation gas to the liquid within the reservoir 624, and increase the efficiency of the humidification process. The diffuser 650 causes a bubbling action of the insufflation gas through the liquid within the reservoir 624. The diffuser 650 can be, for example, a tube structure with holes or perforations, a plate with holes or perforations, a porous member such as a porous stone, or other suitable diffusing component that will allow for the insufflation gas to flow from the inlet port 825, through the liquid within the reservoir 624.

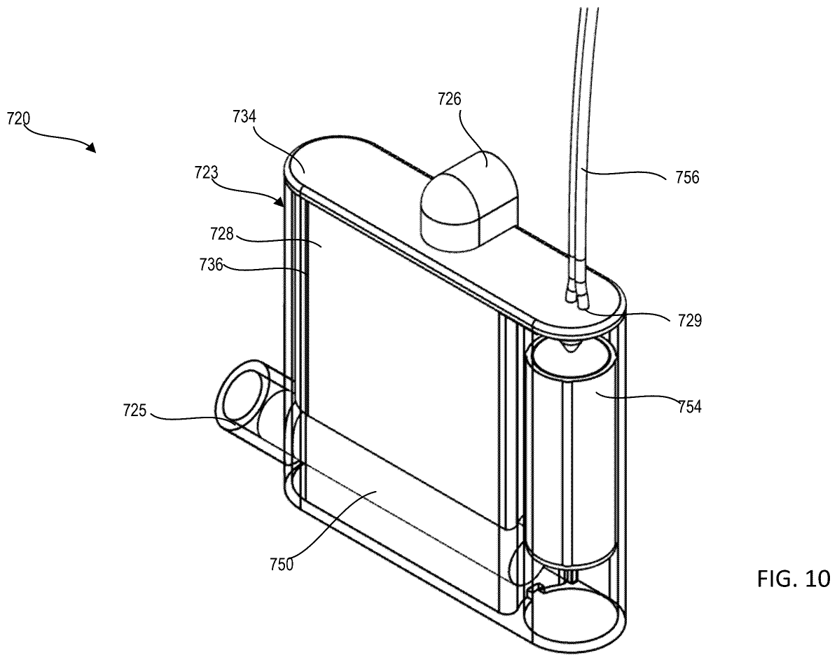

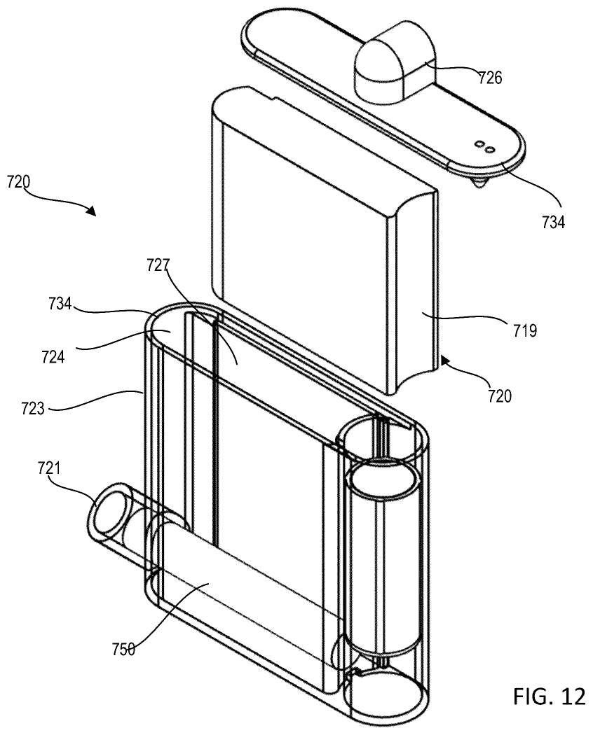

FIGS. 10-12 illustrate a humidifying tube set 720 according to another embodiment. The humidifying tube set 720 can be coupled to an insufflation device as described herein, such as insufflation devices 212 or 412. The tube set 720 includes a tube set housing 723 that defines a reservoir 724 (see FIG. 12) and can be removably received within an interior region (e.g., 215) of the housing (e.g., 214) of an insufflation device (e.g., 212). The housing 723 of the tube set 720 includes a top housing portion 734 and a bottom housing portion 736 that collectively define the reservoir 724. The tube set 720 can include one or more delivery tubes (not shown in FIGS. 10-12) that can be coupled to a delivery instrument (not shown) used to deliver insufflation gas to a body cavity as described herein. The delivery tube(s) can be coupled to an outlet 726 of the tube set housing 723 and be in fluid communication with the reservoir 724. As described above for previous embodiments, the reservoir 724 of the tube set housing 723 can be coupled to a source of insufflation gas (not shown in FIGS. 10-12) such that insufflation gas can be introduced into the reservoir 724 of the tube set housing 723, flow through the reservoir 724, through one or more delivery tubes and into the body cavity of the patient. The source of insufflation gas can be coupled to, for example, an inlet port 725 of the tube set housing 723. In this embodiment, a one-way valve (not shown) is coupled to the inlet port 725 to prevent backflow of insufflation gas and/or liquid within the reservoir 724 out through the inlet port 725.

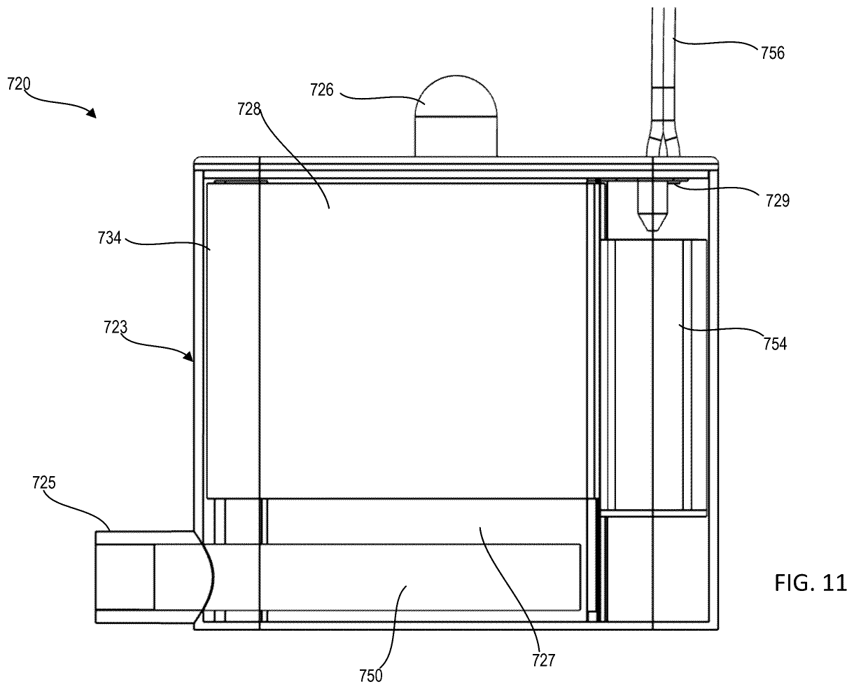

The reservoir 724 can hold a volume of liquid, such as saline or distilled water, which can be heated within the reservoir 724 to heat and humidify the insufflation gas passing through the reservoir 724 and provide humidified insufflation gas to the body cavity. For example, in some cases, it may be desirable to provide insufflation gas at greater than 95% relative humidity to the body cavity. The liquid can be provided via a source of liquid (not shown in FIGS. 10-12) such as a saline bag that can be coupled to a liquid fill port 731 of the housing 723 with fill tubes 756 as shown in FIGS. 10 and 11. A float valve 754 is coupled near the liquid fill port 729 to regulate the flow of liquid into the reservoir 724.

The tube set 720 also includes a conductive member 727 coupled to the housing 723 within the reservoir 724. The conductive member 727 is configured to be heated by energy transferred from an inductive coil of a heater (not shown in FIGS. 10-12) positioned proximate to the housing 723 to heat the volume of liquid and increase a humidity of the insufflation gas flowing through the reservoir 724. As shown in FIGS. 10-12, in this embodiment, the conductive member 727 is disposed on a surface of a sidewall of the bottom housing portion 736. In some embodiments, the conductive member 727 is positioned to be aligned with the inductive coil of the heater of the insufflation device when the tube set 720 is coupled thereto. In this embodiment, the conductive member 727 is a metal plate having a planar configuration. In some embodiments, the conductive member 727 is physically spaced apart from the inductive coil of the heater of the insufflation device. The inductive coil heater (e.g., 216, 416 described above) is configured to heat the conductive member 727 to heat the volume of liquid (e.g., saline or distilled water) within the reservoir 724 of the tube set housing 723.

The tube set 720 also include a filter 728 within the reservoir 724. As described above, the filter 728 can be a single construction of multiple filters, a single filter, or multiple separate filters. As described for previous embodiments, the filter 628 can include one or more filter components 719. In some embodiments, the filter 728 is a corrugated paper construction. In some embodiments, the filter 728 can be a baffle construction. The filter 728 can be constructed with, for example, a nylon, a sponge material, metal mesh, steel wool, or other porous coalescing media that is biocompatible and does not dissolve when exposed to liquid. The filter 728 is configured to transfer a portion of the volume of liquid disposed within the tube set housing 723 to a location within the housing 723 to provide humified fluid within the reservoir 724. For example, as the volume of liquid within the reservoir 724 is heated, the liquid will collect on the filter 728, and as the insufflation gas flows through the reservoir 724, the insufflation gas will be heated and humidified as it passes through the filter 728.

The tube set 720 also includes a diffuser 750 as shown in FIGS. 10-12. In this embodiment, the diffuser 750 is a tubular construction in fluid communication with the inlet port 725. The diffuser 750 can include holes or perforations (not shown) to allow insufflation gas to flow from the inlet port 725, through the holes or perforations, and into the reservoir 724. The diffuser 750 causes a bubbling action of the insufflation gas through the liquid within the reservoir 724, which can increase the contact time of the insufflation gas to the liquid within the reservoir 724, and increase the efficiency of the humidification process. In this embodiment, the diffuser 750 is a separate component coupled within the housing 723. In alternative embodiments, the diffuser 750 can be formed monolithically or integral with the housing 723. In still other alternative embodiments, the diffuser 750 can be, for example, a plate with holes or perforations, or a porous member such as a porous stone, or other suitable diffusing component that will allow for the insufflation gas to flow through and into the liquid within the reservoir 724.

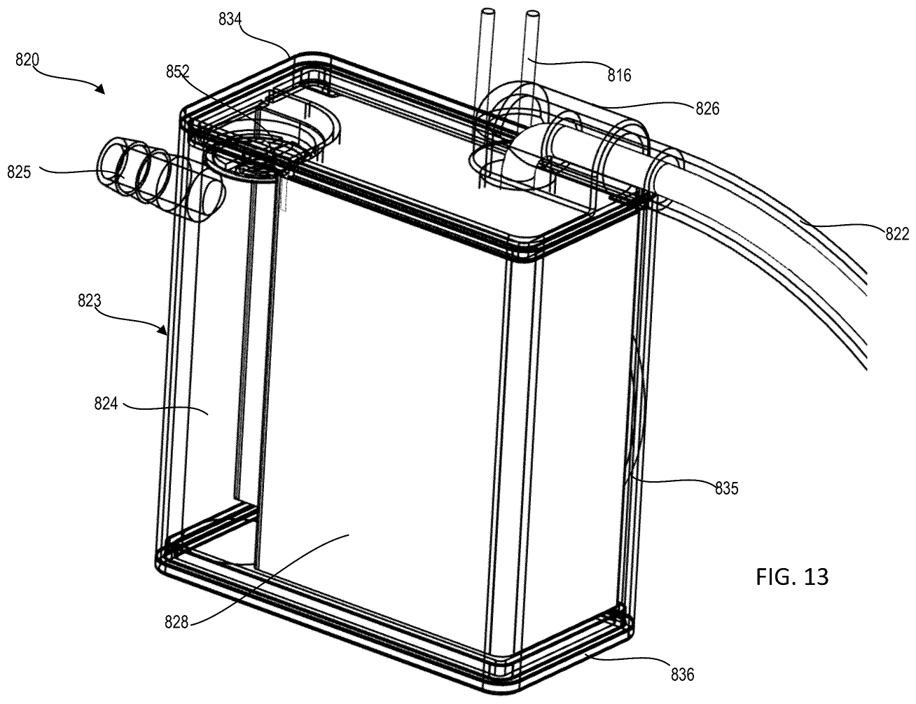

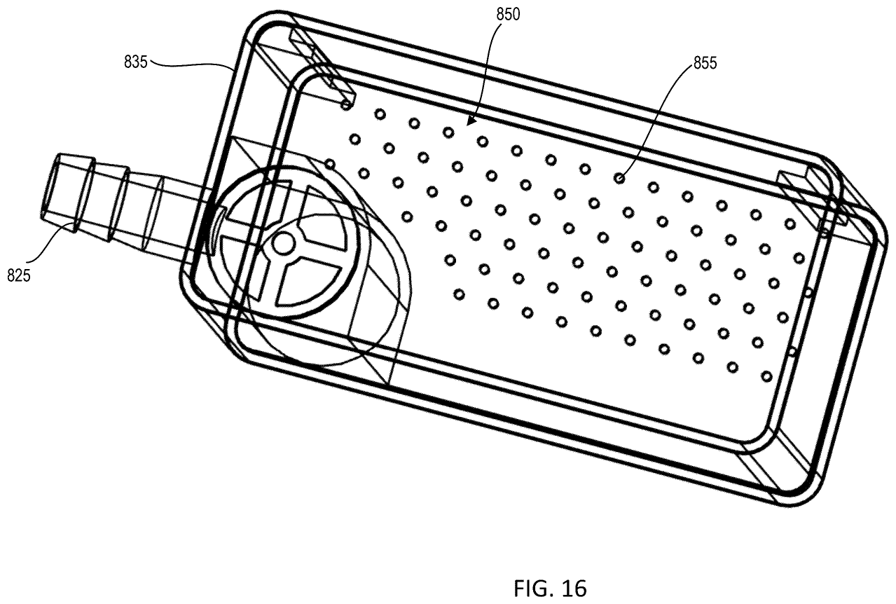

FIGS. 13-16 illustrate a humidifying tube set 820 according to another embodiment. The humidifying tube set 820 can be coupled to an insufflation device as described herein, such as insufflation devices 212 or 412. The tube set 820 includes a tube set housing 823 that defines a reservoir 824 and can be removably received within an interior region (e.g., 215) of the housing (e.g., 214) of an insufflation device (e.g., 212). The housing 823 includes a top housing portion 834, a middle housing portion 835 and a bottom housing portion 836 that collectively define the reservoir 824. The tube set 820 can include one or more delivery tubes 822 that can be coupled to a delivery instrument (not shown) used to deliver insufflation gas to a body cavity as described herein. The delivery tube(s) 822 can be coupled to an outlet 826 of the tube set housing 823 and be in fluid communication with the reservoir 824. As described above for previous embodiments, the reservoir 824 of the tube set housing 823 can be coupled to a source of insufflation gas (not shown in FIGS. 13-16) such that insufflation gas can be introduced into the reservoir 824 of the tube set housing 823, flow through the reservoir 824, through the one or more delivery tubes 822 and into the body cavity of the patient. The source of insufflation gas can be coupled to, for example, an inlet port 825 of the tube set housing 823. In this embodiment, an umbrella valve 852 is operatively coupled to the inlet port 825 to prevent backflow of insufflation gas and/or liquid within the reservoir 824 out through the inlet port 825.

The reservoir 824 can hold a volume of liquid, such as saline or distilled water, which can be heated within the reservoir 824 to heat and humidify the insufflation gas passing through the reservoir 824 and provide humidified insufflation gas to the body cavity. For example, in some cases, it may be desirable to provide insufflation gas at greater than 95% relative humidity to the body cavity. The liquid can be provided via a source of liquid (not shown in FIGS. 13-16) such as a saline bag that can be coupled to a liquid fill port (not shown) of the housing 823 as described above for tube set 720. Alternatively, the liquid can be introduced int the reservoir 824 by removing the top housing portion 834 and introducing the liquid directly into the reservoir 824.

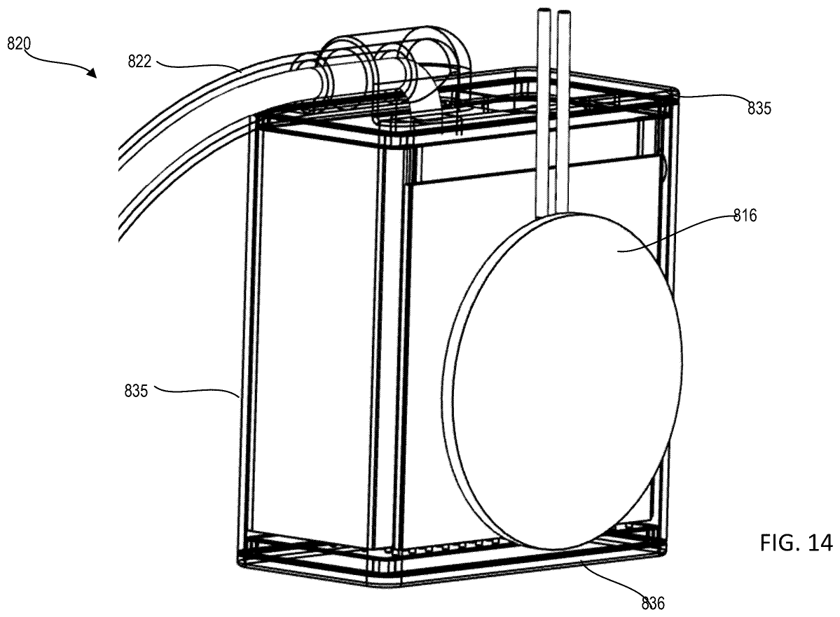

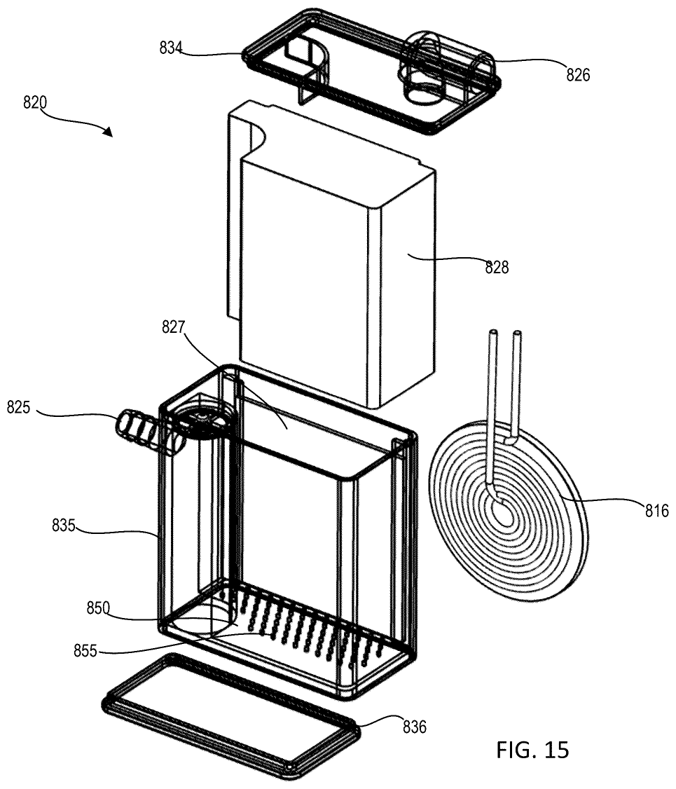

The tube set 820 also includes a conductive member 827 coupled to the housing 823 within the reservoir 824. The conductive member 827 is configured to be heated by energy transferred from an inductive coil 816 (see FIGS. 14 and 15) of a heater positioned proximate to the housing 823 to heat the volume of liquid and increase a humidity of the insufflation gas flowing through the reservoir 824. In this embodiment, the conductive member 827 is a metal plate having a planar configuration. As shown in FIGS. 13-16, the conductive member 827 is coupled to a wall of the middle housing portion 835 of the tube set housing 823 In some embodiments, the conductive member 827 is positioned to be aligned with the inductive coil 816 of the heater of the insufflation device when the tube set 820 is coupled thereto. The inductive coil heater (e.g., 216, 416 described above) is configured to heat the conductive member 827 to heat the volume of liquid (e.g., saline or distilled water) within the reservoir 824 of the tube set housing 823.

The tube set 820 also includes a filter 828 within the reservoir 824. As described above, the filter 828 can be a single construction of multiple filters, a single filter, or multiple separate filter components. In some embodiments, the filter 828 is a corrugated paper construction. In some embodiments, the filter 828 can be a baffle construction. The filter 828 can be constructed with, for example, a nylon, a sponge material, metal mesh, steel wool, or other porous coalescing media that is biocompatible and does not dissolve when exposed to liquid. The filter 828 is configured to transfer a portion of the volume of liquid disposed within the tube set housing 823 to a location within the housing 823 to provide humified fluid within the reservoir 824. For example, as the volume of liquid within the reservoir 824 is heated, the liquid will collect on the filter 828, and as the insufflation gas flows through the reservoir 824, the insufflation gas will be heated and humidified as it passes through the filter 828.

The tube set 820 also includes a diffuser 850 as shown in FIGS. 13-16. In this embodiment, the diffuser 850 is formed monolithically as part of the middle housing portion 835 and includes holes 855 to allow insufflation gas to flow from the inlet port 825, through the holes 855 and into the reservoir 824. The diffuser 850 causes a bubbling action of the insufflation gas through the liquid within the reservoir, which can increase the contact time of the insufflation gas to the liquid within the reservoir 824, and increase the efficiency of the humidification process. In alternative embodiments, the diffuser 850 can be a separate component coupled within the housing 823. In still other alternative embodiments, the diffuser 850 can be, for example, a tubular construction as describe above for diffuser 750. The diffuser 850 can be, for example, any suitable component with holes or perforations, or a porous member such as a porous stone, or other suitable diffusing component that will allow for the insufflation gas to flow through and into the liquid within the reservoir 824.

Inventive aspects disclosed herein may be embodied and implemented in various ways, including computer-assisted, non-computer-assisted, and hybrid combinations of manual and computer-assisted embodiments and implementations. Implementations are merely presented as examples, and they are not to be considered as limiting the scope of the inventive aspects disclosed herein. As applicable, inventive aspects may be embodied and implemented in both relatively smaller, hand-held, hand-operated devices and relatively larger systems that have additional mechanical support.

While various embodiments have been described above, it should be understood that the various embodiments have been presented by way of example only and not limitation. Where methods and/or schematics described above indicate certain events and/or flow patterns occurring in certain order, the ordering of certain events and/or operations may be modified. While the embodiments have been particularly shown and described, it will be understood that various changes in form and details may be made.

For example, any of the tube sets, insufflation systems, or devices described herein (and the components therein) are optionally parts of a telesurgical system that performs minimally invasive surgical procedures, and which can include a manipulator unit, a series of kinematic linkages, a series of cannulas, or the like. Thus, any of the tube sets or insufflation systems described herein can be used in any suitable surgical system

Further, any of the components of the medical instruments described herein can be constructed from any suitable material, such as medical grade stainless steel, nickel alloys, titanium alloys or the like. Further, any of the components described herein can be constructed from multiple pieces that are later joined together.

Although various embodiments have been described as having particular features and/or combinations of components, other embodiments are possible having a combination of any features and/or components from any of embodiments as discussed above. For example, although not shown for all embodiments, any of the embodiments of a tube set described herein can include one or more sensors such as temperature sensors, pressure sensors, one or more valves, such as one-way valves, float valves, etc., diffuser(s), filter(s), etc. Further, aspects have been described in the general context of medical devices, and more specifically surgical instruments, but inventive aspects are not necessarily limited to use in medical devices.

1. A tube set for use in an insufflation device, comprising:
a housing defining a reservoir, an inlet port, and an outlet port, the inlet port and the outlet port each being in fluid communication with the reservoir, the inlet port configured to be coupled to a source of insufflation gas to produce a flow of insufflation gas through the reservoir, the reservoir configured to hold a volume of liquid; and
a conductive member coupled to the housing and configured to be heated by energy transferred from an inductive coil placed proximate to the housing to heat the volume of liquid to increase a humidity of the insufflation gas flowing through the reservoir.

2. The tube set of claim 1, wherein the housing is configured to be removably coupled to an insufflation device.

3. The tube set of claim 2, wherein the conductive member is positioned to be aligned with an inductive coil of the insufflation device.

4. The tube set of claim 1, wherein the conductive member is a plate having a planar configuration.

5. The tube set of claim 1, wherein the conductive member is physically spaced apart from the inductive coil of the insufflation device.

6. The tube set of claim 1, further comprising:
a tube coupled to the outlet port and configured to convey humidified insufflation gas from within the reservoir to a location within a patient's body.

7. The tube set of claim 6, wherein the tube is devoid of a heating element.

8. The tube set of claim 1, further comprising:
a liquid port configured to be coupled to a source of liquid to be introduced into the reservoir.

9. The tube set of claim 1, further comprising:
a filter disposed within the reservoir and configured to transfer a portion of the volume of liquid disposed within the housing to a location within the housing to provide humified fluid within the reservoir.

10. The tube set of claim 1, further comprising:
a diffuser disposed within the reservoir and configured to allow insufflation gas to flow through the diffuser and into the liquid within the reservoir.

11. The tube set of claim 10, wherein:
the diffuser includes holes or perforations to allow insufflation gas to flow through the diffuser.

12. The tube set of claim 10, wherein:
the diffuser is a porous structure configured to allow insufflation gas to flow through the diffuser.

13. The tube set of claim 1, wherein:
the conductive member is disposed within the reservoir and positioned adjacent to a wall of the housing.

14. An insufflation system, comprising:
an insufflator housing configured to removably receive a humidifying tube set couplable within an interior of the insufflator housing, the humidifying tube set including a tube set housing defining a reservoir and a conductive member coupled to the tube set housing;
an inductive coil heater positioned to heat the conductive member of the humidifying tube set when the humidifying tube set is coupled within the insufflator housing;
and
a controller coupled to the inductive coil heater.

15. The insufflation system of claim 14, further comprising:
a temperature sensor positioned within the insufflator housing to measure a temperature of the humidifying tube set when the humidifying tube set is coupled within the insufflator housing.

16. The insufflation system of claim 15, wherein:
the temperature sensor is disposed spaced apart from the humidifying tube set when the humidifying tube set is coupled within the housing.

17. The insufflation system of claim 15, wherein the temperature sensor includes an infrared sensor.

18. The insufflation system of claim 14, wherein:
the conductive member is disposed within the reservoir and positioned adjacent a wall of the tube set housing.

19. The insufflation system of claim 14, wherein:
the inductive coil heater is configured to heat the conductive member of the humidifying tube set to heat a liquid within the reservoir of the humidifying tube set and create a relative humidity within the humidifying tube set of greater than 95%.

20. The insufflation system of claim 17, further comprising:
a source of insufflation gas couplable to an inlet port of the humidifying tube set.

21-22. (canceled)
